(19) United States
(12) Patent Application Publication
(10) Pub. No.: US 2010/0093606 A1
(43) Pub. Date: Apr. 15, 2010

(54) MRSA Bactericidal Topical Gel

(76) Inventor: Robert Stanley Berman, Jupiter, FL (US)

Correspondence Address: Robert S. Berman MD, 19558 Red Gum Trail, Jupiter, FL, 33458, US

(21) Appl. No.: 12/236,246

(22) Filed: Oct. 14, 2008

(51) Int. Cl. A61K 38/14

(52) U.S. Cl. 514/8

(57) ABSTRACT

I have combined Vancomycin at varying concentrations in a Calcium Alginate gel and treated open chronic wounds that cultured out MRSA. The response has been remarkable in the rapid cure of the involved wound. I have arrived at a 15% level of the Vancomycin as being an effective strength. The combined gel, if refrigerated, maintains it's effectiveness over three months. Only one to two applications results in negative cultures.

### BACKGROUND AND SUMMARY

### Technical Field: Medical and Surgical

Methicillin resistant staph aureus is an organism, that is apparent by the name, a resistant and extremely virulent bacteria that is difficult to eradicate. I am a General Surgeon and wound care specialist and found a combination of medications to promptly eliminate the MRSA in open wounds within 24 to 48 hours. My treatment over the past 10 years has never failed to result in negative cultures at one week intervals.

### DETAILED DESCRIPTION OF PREPARATION AND APPLICATION

Vancomycin solution is prepared by diluting the dry form with saline resulting in 1 mg per cc. 15 cc's of the solution is added to 85 cc's of a calcium alginate gel. The resulting gel is then refrigerated and the patient's wound or wounds are treated by applying an ⅛ to ¼ inch layer over the wound and then covered with Adaptic gauze and a dry dressing. The wound is redressed daily using the same medication and dressings. Cultures were taken before the first dressing and generally one week later. The potency of the Gel remains bactericidal even at 3 months if kept refrigerated. The Gel used by itself without the Vancomycin is marketed as Saf-Gel and has the following ingredients: Purified Water USP, Carbomer 940 NF, Triethalamine, Boric Acid NF, Sodium/Calcium Alginate, Potassium Sorbate NF, DMDM Hydantoin and Caboxmethylcellulose USP. The Gel also contributes to wound healing but is not bactericidal.

1. The above titled application “MRSA Bactericidal Topical Gel has proven to be a topical bactericidal combination of medication to eliminate the MRSA infected open wounds in a safe, convenient and economical manner.
